(19) United States
(12) Plant Patent Application Publication
(10) Pub. No.: US 2002/0152526 P1
(43) Pub. Date: Oct. 17, 2002

(54) Lysimachia plant named 'Walgoldalex'

(76) Inventors: Albert Timothy Alan Crowther, Arundel (GB); David Ralph Tristram, Arundel (GB)

Correspondence Address: MARK P. BOURGEOIS, P.O. BOX 95, OSCEOLA, IN, 46561, US

(21) Appl. No.: 09/834,868

(22) Filed: Apr. 14, 2001

(51) Int. Cl. A01H 5/00

(52) U.S. Cl. PLT/263

(57) ABSTRACT

A new cultivar of Lysimachia punctata named ‘Walgoldalex’ that is characterized by it's variegated foliage with green centers and gold margins and leaves that exhibit less distortion than ‘Alexander’.

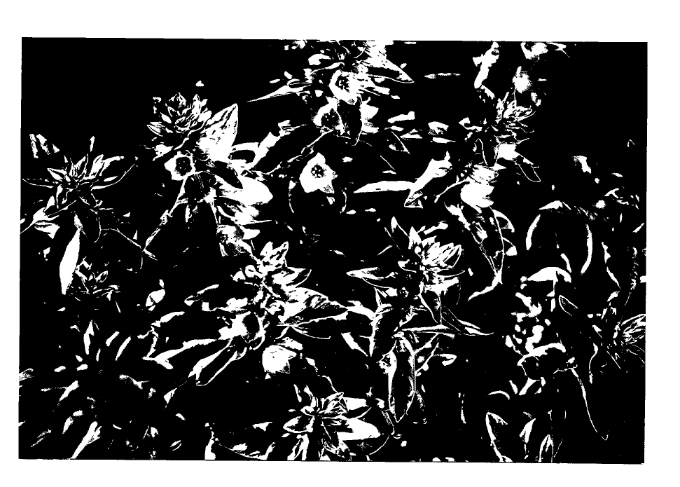

### BACKGROUND OF THE INVENTION

The present invention relates to a new and distinct cultivar of Lysimachia punctata and will be referred to hereafter as ‘Walgoldalex’. Lysimachia punctata is a hardy perennial grown for landscape use.

The new Lysimachia, ‘Walgoldalex’, was discovered in the spring of 1999 by the inventors in a cultivated area at Walberton Nursery, Arundel, W. Sussex, England. ‘Walgoldalex’ was discovered as a naturally occuring golden variegated leaf sport arising from Lysimachia punctata ‘Alexander’ (U.S. Pat. No. 10598)

In the summer of 1998, a leaf was discovered that had a portion of it that was variegated with a deep golden color. Stem cuttings containing the sport were taken and re-isolated until the entire leaf had the desired pattern and the variegation was stabilized.

The new variety of Lysimachia can be characterized by its gold-margined variegation. The parent plant, ‘Alexander’, has margins that are creamy white in color, as opposed to the gold colored margins of ‘Walgoldalex’. The new cultivar is also unique in that the foliage has less distortion to the leaves, a characteristic often exhibited by ‘Alexander’. ‘Walgoldalex’ is similar to ‘Alexander’ in all other aspects such as plant habit, growth rate, and flowering characteristics. ‘Walgoldalex’ is unlike any other Lysimachia punctata, a species that is not naturally variegated and ‘Alexander’ is the only other variegated cultivar that is known to the inventors.

Asexual reproduction of the new cultivar was first accomplished by cuttings in Arundel, W. Sussex, England by the inventors in the spring of 1999. The characteristics of this cultivar have been determined to be stable and are reproduced true to type in successive generations.

### SUMMARY OF THE INVENTION

The following traits have been repeatedly observed and represent the characteristics of the new cultivar. These attributes in combination distinguish this cultivar as a new and unique cultivar of Lysimachia. ‘Walgoldalex’ has not been tested under all possible conditions and phenotypic differences may be observed with variations in environmental, climatic, and cultural conditions:

1. The foliage of ‘Walgoldalex’ is variegated with green centers and gold margins.

2. The leaves of ‘Walgoldalex’ are less distorted during growth in comparison to the leaves of ‘Alexander’.

3. The flowers of ‘Walgoldalex’ are yellow and flowering occurs throughout the summer.

4. ‘Walgoldalex’ is hardy, a vigorous grower and adaptable to a wide range of cultural conditions.

### BRIEF DESCRIPTION OF THE DRAWING

The photograph is of a mature plant of ‘Walgoldalex’. The golden variegated foliage as well as the overall habit is illustrated. The colors in the photograph are as accurate as possible by conventional photography.

### BOTANICAL DESCRIPTION OF THE PLANT

The following is a detailed description of the new cultivar as grown under garden conditions in Arundel, W. Sussex, England. The color determination is in accordance with the RHS Colour Chart of The Royal Horticultural Society, London, England, except where general color terms of ordinary dictionary significance are used.

Botanical classification: Lysimachia punctata ‘Walgoldalex’.

Commercial classification: Hardy Perennial for landscape use.

Parentage: Naturally occurring sport of Lysimachia punctata ‘Alexander’.

General description:

Blooming period.—Blooms from late June-August in temperate climates.

Plant habit.—Upright, moderately branched, more clump-forming than the species.

Height and spread.—30-75 cm in height, 30-60 cm (eventually more) in width.

Hardiness.—Zone 4-8.

Type.—Hardy herbaceous perennial.

Root system.—Fleshy, branched, slightly stoloniferous.

Propagation.—Terminal tip cuttings, division.

Time to initiate roots.—About 10 days at 20° C.

Time to develop roots.—About 14 days at 20° C.

Culture.—Fertile, moisture retentive soils in full sun or part shade.

Diseases and Pests.—No particular sensitivity or resistance observed.

Stems:

Shape.—Round.

Size.—1.3-6 mm in diameter.

Surface.—Pubescent, longitudinal ridges.

Color.—138A to 138C (range).

Branching.—Up to 10 lateral branches/stem.

Internode length.—1 to 4 cm.

Foliage:

Shape.—Ovate.

Division.—Simple.

Apex.—Obtuse.

Base.—Obtuse to cuneate.

Venation.—Venation upper side recessed, especially the mid-vein, venation on lower side prominent.

Margins.—Entire, very finely toothed.

Arrangement.—Opposite, occasionally alternate.

Quantity.—Up to 30 per lateral branch.

Surface.—Pubescent.

Size.—Up to 10 cm in length, and up to 5.0 cm in width.

Petiole dimensions.—3-6 mm in length and 1.5-3 mm in diameter.

Petiole color.—138A.

Color of young foliage.—Upper surface (lower surface); Centers 144A (lower 144B);

Margins.—162B (lower 162B); Intermediate area between the margin and center 138D (lower 138D).

Color of mature foliage.—Upper foliage (lower surface); Centers 146 B to 191B (146C to 191C), margins 161B to 161C (lower 161C), Intermediate area between the margin and center 195B (lower 195B)).

Flowers:

Type.—Single, bell-shaped, flaring from the base, arranged on spikes arising from leaf

Axils.—flowers face outward from all sides of the flower stem.

Fragrance.—None.

Lastingness.—up to 7 days, self cleaning and not persistent.

Quantity.—20 to 30 flowers per spike, typically 3-10 flowers are open at one time. Size: 2.5 cm in depth, 1.3-1.9 cm in height.

Peduncle.—Held at a 30° angle to stem, 1.25-5 cm in length, 3 mm in width, color 138A to 138C.

Petal general description.—5, ovate in shape, dull in appearance with faint reddish longitudinal veining, acute apex, margins edged with glandular hairs.

Petal size.—1.3-1.9 cm in length, 1 cm in width.

Petal color.—Upper surface 12A to 14B, lower surface 12A (opening and fully open)

Calyx general description.—Sepals 5, linear to lanceolate in shape, acute apex, rough, pubescent surface.

Sepal size.—3-5 mm in length, 3 mm in width.

Calyx color.—Upper and lower surface of sepals, centers are 144A, margins are 162B.

Flower bud.—About 1.2 cm in length and about 3 mm in width, 12A in color.

Reproductive organs:

Stamens.—5, anthers are about 1.5 mm in length, about 0.75 mm in width. Pollen color is 21A, abundance is moderate.

Pistil.—1, 3-5 mm in length, stigma color 139B, style color 194B, style length 3-5 mm.

Ovary.—1, 194A-196D in color.

Fruit/Seed.—Low in occurance, 5 valved capsule, seeds 0.15 mm in diameter, 165A in color.

1. A new and distinct cultivar of Lysimachia named ‘Wwalgoldalex’ as described and illustrated.
